(19) United States
(12) Patent Application Publication
(10) Pub. No.: US 2007/0086560 A1
(43) Pub. Date: Apr. 19, 2007

(54) Scatter correction

(75) Inventors: Omid Kia, North Bethesda, MD (US); Arun Singh, North Wales, PA (US); Edward Marandola, Gwynedd, PA (US); Uwe Mundry, Mauldin, SC (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: IMAGING SCIENCES INTERNATIONAL, INC., Hatfield, PA (US)

(21) Appl. No.: 11/543,183

(22) Filed: Oct. 4, 2006

Related U.S. Application Data
(60) Provisional application No. 60/724,244, filed on Oct. 6, 2005.

(51) Int. Cl. H05G 1/60; A61B 6/00; G01N 23/00; G21K 1/12

(52) U.S. Cl. 378/7; 378/19

(57) ABSTRACT

In one embodiment of a method of and apparatus for correcting for scatter, an object, which may be the jaw of a dental patient, is subjected to x-rays or other penetrating radiation. An intensity distribution of the transmitted radiation is detected. A first array of voxel data representing the absorption of the radiation by the object is reconstructed from the detected intensity. A radiation scatter pattern is calculated by forward projection from the first array using one or more point spread functions. The detected intensity is corrected using the calculated radiation scatter pattern. A second array of voxel data representing the absorption of the radiation by the object is reconstructed from the corrected detected intensity.

### CROSS REFERENCE TO RELATED APPLICATIONS

This application claims the benefit of U.S. Provisional Patent Application No. 60/724,244, filed Oct. 6, 2005, which is incorporated herein by reference in its entirety.

### BACKGROUND

The invention relates to the extraction of useful information from the examination of objects with penetrating radiation, and especially to the generation of more accurate and precise data from the results of x-ray scans and images by removing or reducing the effects of scattered, non-imaging radiation.

The viewing of objects, including parts of the human anatomy, by the use of x-rays and other forms of penetrating radiation is known. In the case of x-rays, the radiation is directed at the object from one side, and the part of the radiation that penetrates the object is detected at the opposite side. An image may thus be obtained in which parts of the object that are more absorbent of x-rays, typically more dense parts of the object, appear as darker shadows, either directly on an x-ray sensitive film or by detecting the x-rays electronically and generating an image using a computer. Alternatively, in a computed tomography (CT) system, a series of x-ray images of a target are taken with the direction from the source to the detector differently oriented relative to the target. From these images, a three-dimensional representation of the density of x-ray absorbing material in the target may be reconstructed. Other methods of generating a three-dimensional dataset are known, including magnetic resonance imaging, or may be developed hereafter. From the three-dimensional data, a tomogram, which is a section in a desired plane, may be generated.

However, real objects do not simply absorb or transmit x-rays and other forms of penetrating radiation, but also scatter the radiation. In the simplest scenario, the scattered radiation produces a uniform fog of non-imaging radiation on the detectors, which reduces the contrast of the image, and makes determination of the absolute value of the x-ray density of the tissue or other material making up the object difficult. It has been proposed to measure x-ray intensity near the edges of a detector array, outside the direct beam from the x-ray source, and to generate a scatter pattern by interpolating from these measurements. However, such an approach can only correct for scatter that is uniform, or uniformly varying, across the detector area.

In practical applications, however, the x-rays are differently scattered by different materials, and may be scattered specifically by boundaries between materials and other structures. This leads not only to “cupping artifact,” a non-uniform scatter in which the radiation intensity is lowest at the center of the image area, but also to smaller artifacts that may obscure, or may be mistaken for, image detail.

### SUMMARY

According to one embodiment of the invention, there is provided a method and system for correcting for scatter, comprising subjecting an object to penetrating radiation, detecting the intensity of the transmitted radiation, reconstructing from the detected intensity a first array of data representing the absorption of the radiation by the object, calculating by forward projection from the first array using one or more point spread functions a radiation scatter pattern, correcting the detected intensity using the calculated radiation scatter pattern, and reconstructing from the corrected detected intensity a second array of data representing the absorption of the radiation by the object.

In a preferred embodiment, the process of calculating, correcting, and reconstructing may be repeated one or more times.

According to another embodiment of the invention, there is provided a method and system for correcting for scatter, comprising subjecting a target object to penetrating radiation, detecting the intensity of the transmitted radiation, correcting the detected intensity using a scatter pattern for a known object similar to the target object, and reconstructing from the corrected detected intensity an array of data representing the absorption of the radiation by the object.

According to a further object of the invention, there is provided a method and system for generating a scatter pattern from a known object, comprising subjecting the known object to penetrating radiation, detecting the intensity of the transmitted radiation, calculating the transmitted radiation pattern with no scatter, and subtracting the calculated radiation pattern from the detected intensity.

In a preferred embodiment, the target object is the head, or part of the head, of a human patient, for example, the mandibular and/or maxillary regions of the head of a dental patient, and the known object is an artificially created dummy head or partial head.

The invention also provides computer software arranged to correct for scatter in accordance with the method of the invention, and computer-readable media containing such software. The software may be written to run on an otherwise conventional computer processing tomographic data.

The invention also provides data processed by the methods and systems of the invention.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a schematic view of apparatus for generating a tomographic image.

FIG. 2 is a flow chart of a first form of a method according to the invention.

FIG. 3 is a flow chart of a second form of a method according to the invention.

### DETAILED DESCRIPTION

Reference will now be made in detail to various embodiments of the present invention, examples of which are illustrated in the accompanying drawings.

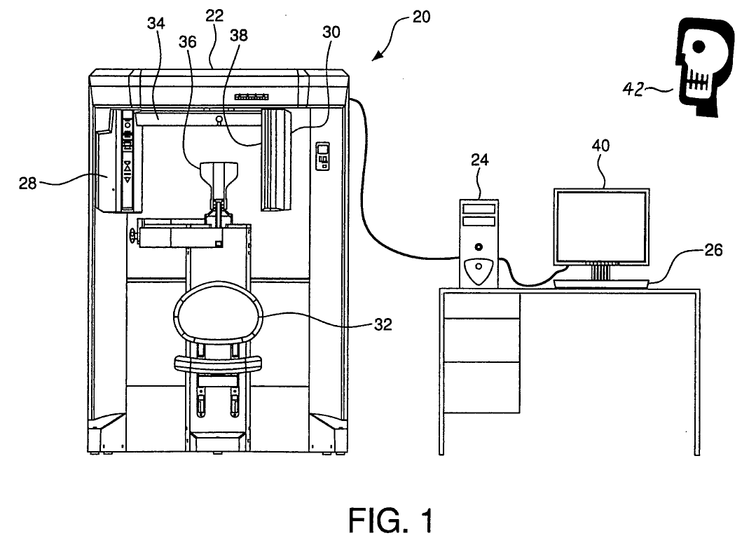

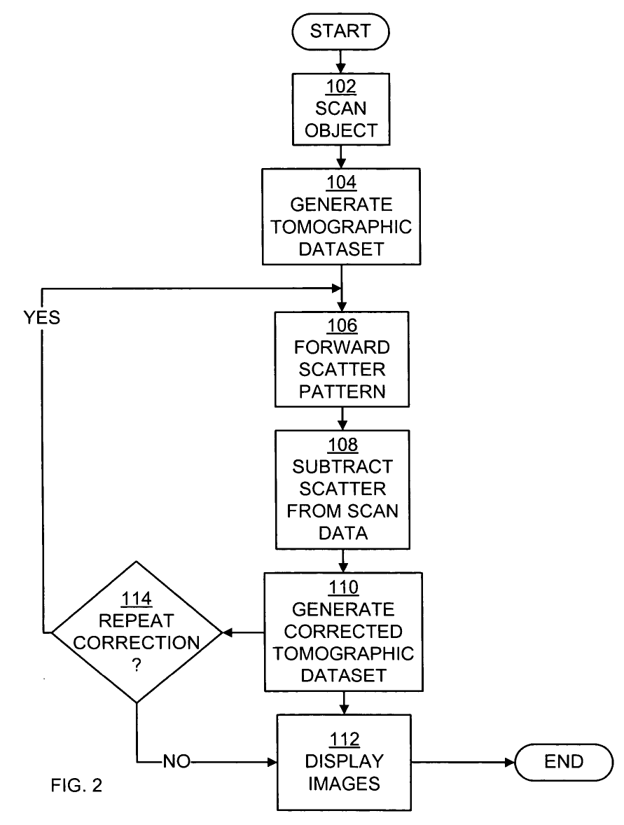

Referring to the drawings, and initially to FIGS. 1 and 2, one form of tomographic apparatus according to an embodiment of the invention, indicated generally by the reference numeral 20, comprises a scanner 22 and a computer 24 controlled by a console 26 with a display 40. The scanner 22 comprises a source of x-rays 28, an x-ray detector 30 including an array of sensors 38, and a support 32 for an object to be imaged. In an embodiment, the scanner 22 is arranged to image the head, or part of the head, of a human patient (not shown), especially the jaws and teeth. The support 32 may then be a seat with a rest or restrainer 36 for the head or face (not shown) of the patient. The x-ray source 28 and detector 30 are then mounted on a rotating carrier 34 so as to circle round the position of the patient's head, while remaining aligned with one another. The x-ray detector 30 then records a stream of x-ray shadowgrams of the patient's head from different angles. The computer 24 receives the x-ray image data from the scanner 22, and calculates a 3-dimensional spatial distribution of x-ray density.

Although a head x-ray scanner is shown by way of example in FIG. 1, the present method is not only applicable to x-ray head scanning, but pertains to other digital imaging devices, including whole body CT, digital x-ray, etc. The method may also be applied to x-ray imaging of objects other than medical patients, in any circumstances where precise determination or discrimination of x-ray density is desired.

The imaging of the patient's head and calculation of the spatial distribution may be carried out by methods and apparatus already known in the art and, in the interests of conciseness, are not further described here. Suitable apparatus is available commercially, for example, the i-CAT Cone Beam 3-D Dental Imaging System from Imaging Sciences International of Hatfield, Pa.

Soft tissue may be distinguished from hard tissue by density. The contrast between flesh and bone in the human body is sufficiently definite that a clear distinction is easily made. For example, in normalized Hounsfield Units, water has a value of 0 and other materials have values from −1000 (wholly transparent to x-rays) to +3000 or higher (wholly opaque to x-rays). Fat then typically has a density just below 0 HU, soft tissue typically has a density between 0 and 100 HU, and bone typically has a density of >100 HU. A threshold density for distinguishing soft tissue from bone may then be set at, for example, 100 HU. Exact values may vary because Hounsfield Units are not perfectly objectively quantified, or because of differing preferences as to the treatment of materials having a density close to the threshold. The presence of cupping artifact or other non-uniformity in the measured radiation intensity over the width of the detector array may make reliable discrimination more difficult. The difficulty may be exacerbated if finer discrimination, for example, between different forms of soft tissue or between different grades of bone, is required.

In medical Computed Tomography, the voxels of the tomographic dataset are typically brick-shaped with a square footprint, with sides typically in the range of 0.5 mm to 1 mm. An accepted standard for definition is that a contrast difference of 0.25%, or 2.5 HU, between adjacent voxels should be resolvable when the density edge is at least 2.5 mm in length. For fine detail, a spatial resolution of 0.5 mm to 1 mm may be used. Because dental surgeons require very fine detail of small areas, dental tomography apparatus is available with a spatial resolution in the range of 0.1 mm to 0.4 mm. The presence of small artifacts within the image may hinder the resolution or recognition of structure.

Referring now to FIG. 2, in one example of a process according to the invention, in step 102, the x-ray detector 30 records x-ray data of the patient's head from different angles, and in step 104 the computer 24 receives the x-ray image data from the scanner 22 and calculates a tomographic dataset representing a 3-dimensional spatial distribution of x-ray density.

In step 106, the computer 24 generates a scatter pattern by forward projecting the scattered x-rays from the voxels of the tomographic dataset to the detector array, for each of the original images. In one embodiment of step 106, representative voxels within the dataset are selected as point sources of scattered radiation. Each point source is assigned a power and spread on the basis of local contrast and density information and the incident x-ray power density at the point. The three-dimensional point spread function is then mapped to the receptor using the assigned radial dispersion from the point and using the volumetric absorption of the regions between the point source and the receptor. Because the spread is calculated in three dimensions, it is found that superior results can be achieved in comparison with prior art systems in which a single ray is traced from a source point to the receptor and then blurred. The number of scatter point sources used may be selected in dependence on the desired accuracy and the granularity of the scatter generating anatomy.

Because of the circularly-symmetrical nature of the point spread function, the three-dimensional spread from a specific scatter point source may be applied to neighboring voxels provided that both the scatter properties of the voxels and the volumetric absorption of the regions between the voxels and the receptor are sufficiently uniform. In particular, a spherical region of uniform properties may be treated as if it were a single large “point.” Scattering at a boundary may need to be treated separately from the scattering in the bulk material on either side of the boundary. Since scatter is typically a low spatial frequency phenomenon, local contrast information can be used with low resolution, and therefore a small number of scatter points can typically be used to describe the scatter-generating anatomy. Depending on the known or assumed properties of the different tissues making up the head, a single point spread function may be applied overall, with only the power varying, or different point spread functions may be used depending on the tissue at the source point, as recognized from the initially estimated density.

Although increasing the number of independently calculated scatter point sources increases the accuracy of the scatter pattern calculated, there is a point of diminishing returns. Further simplification can be achieved by limiting scatter generation to within a certain distance of the detector or absorption length.

In step 108, the computer 24 subtracts the scatter pattern generated in step 106 from the image data recorded in step 102 to produce corrected image data representing the images that would be detected by the x-ray detector 30 if the scatter were not present. In step 110, the computer 24 calculates a corrected tomographic dataset using the corrected image data from step 108. Alternatively, in step 106 the computer 24 may generate a notional tomographic dataset directly representing the spurious data resulting from scatter that is present in the initial tomographic dataset from step 104. The notional tomographic dataset can then be subtracted directly from the initial tomographic dataset. The latter approach is faster, if it is desired merely to correct general macro level artifacts such as cupping/capping. However, the process of correcting the image data, although computationally more intensive and therefore slower, is believed to allow finer resolution in the scatter correction, and therefore greater enhancement of detail within the data.

From step 110, the process may proceed to step 112, and display at the console 26 images based on the corrected tomographic dataset, for example, tomographic slice images or synthesized shadowgrams presenting a view of the patient's head requested by a user. Programs for generating such images from a tomographic dataset are commercially available, and in the interests of conciseness will not be described here.

Alternatively, from step 110 the process may proceed to step 114, where a decision is made whether to repeat the correction in order to obtain a further improved dataset. Step 114 may cause the correction to be repeated a preselected number of times. The number of times may be determined by obtaining a substantially scatter-free control dataset of a phantom 42 or other test object, scanning the same phantom on the scanner 22 to provide a test dataset, and determining experimentally how many iterations of steps 106, 108, and 110 give the closest match between the control dataset and the test dataset. The control dataset may be obtained, for example, by scanning the phantom 42 on a high-quality fan beam CT system with established HU accuracy, or by using a phantom with known density regions that is physically free of any cupping or other scatter related phenomenon, has known geometric shapes and has known constant density regions, and calculating a control dataset that exactly matches the known phantom. A further alternative is to generate the control dataset by scanning the phantom 42 using a focused grid to block scattered rays. If a repetition is desired, the process loops back to step 106, and repeats steps 106, 108, and 110. If a further repetition is not desired, the process proceeds to step 112 and generates the requested images using the corrected dataset from the last iteration of step 110.

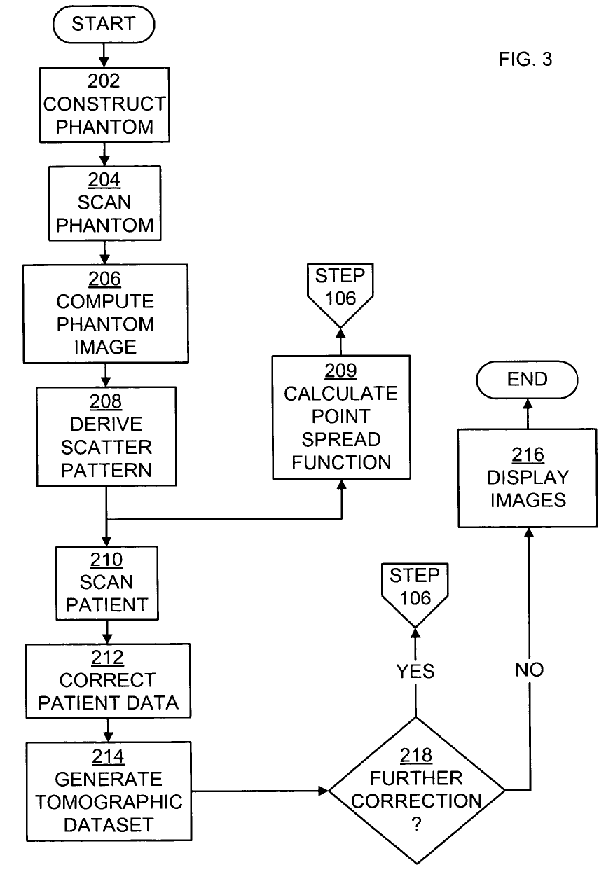

Referring now to FIG. 3, in a second embodiment of a process according to the invention, in step 202 a head phantom 42 is constructed. The phantom 42 is an artificial head or partial head of known dimensions and known x-ray density at each point. The phantom 42 comprises at least the part of the head that will be within the x-ray beam during imaging. The phantom 42 comprises at least components of different densities corresponding to the bone, soft tissue, and teeth of a typical human head. Several phantoms 42 of different sizes and/or shapes, corresponding to different typical human heads may be constructed. In one example, the phantom 42 may consist of an actual human skull, with artificial soft tissues of known x-ray densities and distribution. The construction of x-ray phantoms is a well-known and well-understood procedure, and in the interests of conciseness will not be further described here.

In step 204, the x-ray detector 30 records x-ray data of the phantom 42 from different angles. In step 206, the computer 24 computes, from the known properties of the phantom 42, how the x-ray data should appear in the absence of scattering. Although in the interests of simplicity step 206 is shown as following step 204, in reality step 206 can be carried out as soon as the design of the phantom 42 and the planned alignment of the x-ray exposures are known, and may be carried out independently of step 204 on a different computer.

In step 208, the computer 24 subtracts the computed x-ray data obtained in step 206 from the actual x-ray data obtained in step 204, and determines the scattering component of the actual data. Where several phantoms 42 have been constructed, each phantom may be processed in steps 204 through 208. The phantom 42, or each of the phantoms 42, may be processed in steps 204 through 208 with different alignments of the phantom relative to the scanner 22.

In one alternative, the scatter pattern obtained in step 208 by comparing the calculated and actual datasets from steps 204 and 206 is used in step 209 to calculate the point spread function or functions. For this purpose, a phantom 42 simpler in form than a human head may be preferred. The phantom 42 may be deliberately designed to present the important scattering phenomena in a form that is easy to analyze. For example, the phantom 42 may be a small sphere of material of known x-ray density and scattering power, in order to generate a very simple scattering pattern. A more complex object may then be represented by assembling a suitable array of spheres, and the scatter pattern of the complex object may be calculated by summing the scatter patterns derived from the point spread functions of the individual spheres. The point spread functions from step 209 may then be passed to step 106 to generate scatter patterns for a tomographic dataset obtained in step 104.

Alternatively, a phantom 42 may be scanned as if it were an actual patient's head, in order to test how well the correction process is working.

Alternatively, in step 210, the x-ray detector 30 records x-ray data of an actual patient's head from different angles. In step 212, the computer 24 receives the x-ray image data from the scanner 22 and subtracts the scattering component data determined in step 208 to produce corrected image data. Where more than one set of scattering component data are available, the set from the phantom 42 corresponding most closely in size, shape, and orientation to the head of the actual patient is used. The appropriate phantom 42 may be selected by pattern-recognition on the raw patient data to identify the head size and position, optionally augmented by scaling the nearest phantom scattering data, or interpolation between two phantom datasets or union of different phantoms or anatomy models, to achieve a better fit than can be obtained from any single phantom dataset. Additional parameters of the phantom 42 may also be matched and/or adjusted to those of the patient's head.

Although in the interests of simplicity the phantom 42 and the actual head are described as being scanned successively on the same scanner 22, that is not necessary. The scanner 22 used for scanning actual patients may be supplied with phantom scatter data, or a library of phantom scatter datasets, previously generated on a scanner with similar geometry, preferably a scanner of the same make and model.

In step 214, the computer 24 calculates a tomographic dataset representing a 3-dimensional spatial distribution of x-ray density from the corrected image data.

From step 214, the process may proceed to step 216, and display at the console 26 images based on the corrected tomographic dataset, for example, tomographic slice images or synthesized shadowgrams presenting a view of the patient's head requested by a user.

Alternatively, after step 214 the process may proceed to step 218 to determine whether further correction is required. If so, the dataset may be further corrected, for example, by proceeding to step 106 of FIG. 2.

Various modifications and variations can be made in the present invention without departing from the spirit or scope of the invention. Thus, it is intended that the present invention cover modifications and variations of this invention provided they come within the scope of the appended claims and their equivalents.

For example, where the patient's mouth contains metal fillings, implants, or the like, a phantom 42 cannot usually duplicate those metal objects, and the method of step 212 is not applicable to those metal objects. To the extent that metal objects cause scattering that can be represented by a point spread function, the scattering can be corrected by the method of FIG. 2. However, the major effect of metal objects in the patient's mouth is the phenomenon known as “metal artifacts.” Although the term “scatter” is used interchangeably to describe both the diffuse scattering of x-rays by ordinary tissue and the formation of metal artifacts, the two phenomena are very different. The “metal artifacts” result primarily from the loss of data where an opaque metal object conceals structure in line with the metal object. It is presently preferred to eliminate metal artifacts separately by other techniques, where such elimination is desired. An example of such a technique is the Metal Artifact Reduction algorithm commercially available from Exxim Computing Corporation, of Pleasanton, Calif. For other examples see, for example, Randall V. Olsen et al., Metal Artifact Reduction Sequence: Early Clinical Applications, Radiographics. 2000; 20:699-712; T. Rohlfing et al., Reduction of Metal Artifacts in Computed Tomographies for the Planning and Simulation of Radiation Therapy, “CAR'98, Computer Assisted Radiology and Surgery”, Elsevier Science, 1998, pp. 57-62; S. H. Kolind et al., Quantitative evaluation of metal artifact reduction techniques, J Magn Reson Imaging. 2004 September; 20(3):487-95.

For example, instead of scanning an entire head phantom 42, separate phantoms may be provided for different components of the head, such as mandible, maxilla, cheek, teeth, fillings, metal inserts, vertebrae, and so on. These components may then be individually pattern-matched to the uncorrected tomographic dataset. By adjusting the size, position, orientation, and other parameters of each component individually, a more exact match to the actual head can be achieved, although at the expense of some additional computation.

For example, FIG. 1 shows that the computer 24 on which the process of FIG. 2 and/or FIG. 3 is running is connected to the scanner 22. A single computer 24 may both control the scanner 22 and run the processes of FIGS. 2 and 3. Alternatively, part or all of the process may be carried out on a separate computer. The data from the scanner 22 may be transferred from computer to computer in a convenient format, for example the DICOM format, at a convenient stage of the process. The data may, for example, be transferred directly from computer to computer or may, for example, be uploaded to and downloaded from a storage server.

As noted above, the processing of phantom data to determine the density patterns of new phantoms, or to generate scatter patterns from phantoms of known density distribution, may be carried out on the same or a different scanner 22 and/or on the same or a different computer 24. The point spread functions depend primarily on the properties of the scattering tissue and the spectrum of the x-rays, and may be generated or verified on any scanner having a suitable spectrum, although it may be preferred to generate or verify the point spread functions using a scanner similar to the scanner on which the scans of actual patients or other objects will be carried out. Where a number of substantially identical scanners 22 are being manufactured, the phantoms 42 may be scanned once, under highly controlled conditions, by the manufacturer and each scanner 22 may be supplied with a library of copies of the phantom scatter datasets and/or of the point spread functions. Where otherwise similar scanners 22 are to be used for different purposes, different libraries maybe supplied.

Varying proportions of the scattered radiation may be removed, depending in part on the number of iterations of the loop in FIG. 2. In theory, removing all of the scatter may be ideal. In practice, however, each iteration causes some loss of image data, and in a practical application the optimum final image may be obtained by removing only part, for example, 50% to 70%, of the scatter.

What is claimed is:

1. A method of correcting for scatter, comprising:
subjecting an object to penetrating radiation;
detecting an intensity distribution of the transmitted radiation;
reconstructing from the detected intensity a first array of voxel data representing the absorption of the radiation by the object;
calculating by forward projection from the first array using one or more point spread functions a radiation scatter pattern;
correcting the detected intensity using the calculated radiation scatter pattern; and
reconstructing from the corrected detected intensity a second array of voxel data representing the absorption of the radiation by the object.

2. A method according to claim 1, further comprising repeating the process of calculating, correcting, and reconstructing one or more times.

3. A method according to claim 1, wherein detecting an intensity distribution comprises detecting an amount of radiation received at an array of detectors.

4. A method according to claim 1, further comprising generating and displaying an image representing the object from the second array of voxel data.

5. A method of correcting for scatter, comprising:
subjecting a target object to penetrating radiation;
detecting the intensity of the transmitted radiation;
correcting the detected intensity using a scatter pattern for a known object similar to the target object; and
reconstructing from the corrected detected intensity an array of data representing the absorption of the radiation by the object.

6. A method according to claim 5, wherein the target object is at least part of a human head, and the known object is an artificially created phantom head or partial head.

7. A method according to claim 6, wherein the target object comprises at least part of the mandibular and/or maxillary regions of the head of a dental patient.

8. A method according to claim 5, further comprising generating and displaying an image representing the object from the array of data.

9. A method for generating a scatter pattern from a known object, comprising:
subjecting the known object to penetrating radiation;
detecting an intensity distribution of the transmitted radiation;
calculating a pattern of radiation transmitted by the object with no scatter; and
subtracting the calculated transmitted radiation pattern from the detected intensity distribution.

10. A computer program to cause a computer to correct data for scatter, comprising instructions to cause the computer to:
receive data representing an intensity distribution of radiation transmitted by an object subjected to penetrating radiation;
reconstruct from the detected intensity a first array of voxel data representing the absorption of the radiation by the object;
calculate by forward projection from the first array using one or more point spread functions a radiation scatter pattern;
correct the detected intensity distribution using the calculated radiation scatter pattern; and
reconstruct from the corrected detected intensity a second array of voxel data representing the absorption of the radiation by the object.

11. A computer program according to claim 10, further comprising instructions to cause the computer to repeat the process of calculating, correcting, and reconstructing one or more times.

12. A computer program according to claim 10, wherein the instructions to detect an intensity distribution comprise instructions to detect an amount of radiation received at an array of detectors.

13. A computer program according to claim 10, further comprising instructions to cause the computer program to generate and display an image representing the object from the second array of voxel data.

14. A computer program according to claim 8 on a machine-readable medium.

15. A computer program to cause a computer to correct data for scatter, comprising instructions to cause the computer to:
subject a target object to penetrating radiation;
detect the intensity of the transmitted radiation;
correct the detected intensity using a scatter pattern for a known object similar to the target object; and
reconstruct from the corrected detected intensity an array of data representing the absorption of the radiation by the object.

16. A computer program according to claim 15, further comprising instructions to cause the computer program to generate and display an image representing the object from the array of data.

17. A computer program according to claim 15 on a machine-readable medium.
